(19) United States
(12) Patent Application Publication
(10) Pub. No.: US 2025/0195337 A1
(43) Pub. Date: Jun. 19, 2025

(54) Barium Silicate Radiopaque Dental Cements

(71) Applicant: CAO Group, Inc., West Jordan, UT (US)

(72) Inventor: Steven D. Jensen, South Jordan, UT (US)

(73) Assignee: CAO Group, Inc., West Jordan, UT (US)

(21) Appl. No.: 18/986,474

(22) Filed: Dec. 18, 2024

Related U.S. Application Data
(60) Provisional application No. 63/611,663, filed on Dec. 18, 2023.

(51) Int. Cl. A61K 6/76; A61K 6/69

(52) U.S. Cl. CPC A61K 6/76; A61K 6/69

(57) ABSTRACT

A dental cement with improved radiopacity and delivery methods may feature active barium silicate compounds such as mono-barium silicate, dibarium silicate, and tribarium silicate. The barium silicate compounds are manufactured and then mixed into a paste with an anhydrous carrier and stored in a syringe. Storage in the syringe maintains the paste in an anhydrous state until applied to a treatment site, when water and moisture may be naturally or artificially applied to the paste and begin the curing process. The cement may be use in both dental and orthopedic fields.

### CROSS-REFERENCES TO RELATED APPLICATIONS

The present invention claims priority to prior filed U.S. Provisional Application No. 63/611,663, filed on Dec. 18, 2023, and incorporates the same by reference herein in its entirety.

### FIELD OF THE INVENTION

The present invention relates to the field of dentistry and more particularly relates to an improved radiopaque and anti-microbial dental cement utilizing barium silicate.

### BACKGROUND OF THE INVENTION

There is a need in the medical and dental industry for a syringe-delivered bio-inorganic cement that is compatible with both bone and teeth. Current dental cements are delivered in the form of an anhydrous paste that cures to a solid mass by reaction with water to form a mineral complex/hydrate. Contemporary bio-inorganic cements, also known in the marketplace as “bio-ceramic dental cements,” comprise mainly of calcium and strontium silicates, especially di/tri calcium and strontium silicate cement. These cements are not sufficiently radiopaque, so they require additional radiopaque compounds such as tantalum, bismuth, and zirconium to be added simply for the ability to be radiopaque—these compounds do nothing else for the cement itself. Also, these additional ingredients can, in some degree, create toxicity, corrosion or reactivity issues when placed long term into bone or teeth. Therefore, a balance must be reached when using current bio-ceramic dental cements with added radiopaque compounds so that the resultant cement will be sufficiently radiopaque without creating a cement that this therapeutically detrimental to the patient or reduces the useful life of the reconstruction itself.

The present invention is a dental cement which has greater radiopacity. The present invention departs from the prior art and utilizes the barium cation so that the bio-inorganic cement itself is sufficiently radiopaque and requires no additional radiopaque additives. The present invention utilizes barium silicate compounds, more specifically mono-barium silicate, dibarium silicate, and tribarium silicate and any combination thereof to form these cements.

### SUMMARY OF THE INVENTION

In view of the foregoing disadvantages inherent in the known types of dental cements, an improved dental cement may the following objectives: that it be radiopaque without resorting to compound additives, that it may be manufactured into a flowable paste that can be administered by syringe, and that it be simple and economical to manufacture and administer. As such, a new and improved dental cement may comprise some form of barium silicate, in the form of barium silicate, dibarium silicate, or tribarium silicate, in order to accomplish these objectives.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for description and should not be regarded as limiting.

As such, those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions insofar as they do not depart from the spirit and scope of the present invention.

### DESCRIPTION

A preferred embodiment of the radiopaque dental cements is herein described. It should be noted that the articles “a,” “an,” and “the” as used in this specification, include plural referents unless the content clearly dictates otherwise.

Barium silicates are far superior to calcium and strontium silicate cements because they require no additional radiopaque additive for the cement to be visible radiographically i.e., through x-rays. When tribarium silicate is mixed with water to from a paste, the paste will eventually react to form a hardened cement at a much faster rate than one formed of dibarium silicate. Mono-barium silicate is more or less nonreactive, so the curing rate of any dental cement utilizing barium silicate compounds can be controlled by blending various combinations of each type of silicate to either speed up or slow down the cure rate. The present invention prefers blends of dibarium silicates and tribarium silicates because they create stronger and more resilient cements.

To make the barium silicate compounds, 153.3 g of Barium oxide may thoroughly be mixed with 60 g of precipitated silica and thereafter blended with distilled water to form a paste; the resulting paste was ground in a large mortar and pestle. The paste may then be allowed to dry at 55° C. to remove any excess water, overnight usually being a sufficient time. The resulting dried mass may then be calcined at 1500° C. in a furnace for approximately 8 hours. The resultant clinker will be white in color and composed of dibarium and tribarium silicates. The clinker may then be ground again into a fine powder of dibarium and tribarium silicates. The resulting blend may then be mixed into a paste with the appropriate amount of water and allowed to hydrate hardened into a solid form.

The desired delivery device of the present invention is through a syringe so that the dental or medical practitioner can simply express the material through a desired orifice of a syringe tip to the treatment site. Dibarium and tribarium silicates must be isolated from moisture/humidity until ready to use by the practitioner or they will harden prematurely. Therefore, the barium silicate cement is blended with an anhydrous organic carrier or solvent to form an anhydrous paste which is then loaded into an airtight syringe until the time of use. The carriers of choice include but are not limited to polyethylene glycols, glycerin, propylene glycol and any other useful anhydrous solvent. The anhydrous paste may be directly applied to a treatment site through the syringe. After it is delivered to the treatment site, the anhydrous barium silicate paste is exposed to water and humidity, and it then absorbs water/moisture and eventually cures into a solid.

Another desirable addition to the present invention is to enable the cement to have long-term anti-microbial properties. The preferred antimicrobial additive of the present invention is Zinc pyrithione, although any other suitable anti-microbial agent would work as well. The addition of a long-term antimicrobial compound would also benefit prior art cements, such as (mono, di, tri) calcium/strontium silicate dental cements, especially with the addition of zinc pyrithione. The addition and benefits of a long-term anti-microbial compound to the present invention and prior art cements is within the scope of this patent.

In general, a long-term anti-microbial compound is defined as maintaining at least some measurable anti-microbial reduction after at least one year after being placed into teeth or bone. Zinc pyrithione is an excellent anti-microbial agent that targets a broad spectrum of candidates such as bacteria, yeasts, fungi, etc. with an effective dose being as low as 0.05% by weight of a composition. The preferred range of zinc pyrithione for porous compositions, such that water can move freely throughout the cured composition, is from 0.05%-0.1% by weight. The preferred range of zinc pyrithione for semi-porous compositions, where water can at least absorb into the cured composition, is from 0.1%-1.0% by weight. The preferred range of zinc pyrithione for hydrophobic compositions, where water cannot infiltrate the composition and will only interact with the surface of the cured composition, is from 1.0%-10.0% by weight.

A clinical application of a preferred embodiment of the present invention utilizes a barium silicate cement comprising a majority of tribarium silicate and dibarium silicate, whereby the cement is provided radiopacity by the molecule barium. The barium silicate composition is then blended with an anhydrous carrier forming a deliverable paste. The anhydrous carrier provides a barrier against moisture that could initiate premature curing before it can be used by the practitioner. Alternatively, zinc pyrithione can be added to the dental paste in appropriate concentrations, providing a long-term anti-microbial effect in order to reduce or eliminate opportunistic infections. The cement composition is then loaded into a sealed syringe that becomes the storage device that protects the contents from the ingression of moisture over the shelf-life of the product. Finally, when the end-user, such as a dental or orthopedic practitioner, requires the use of a tooth or bone cement in order to fill, for example an extirpated root canal, they merely need to remove the end cap and attach a delivery tip of their choice and physically deliver the cement to the treatment site. The cement is then naturally exposed to the moisture within the root canal and from the environment will eventually cure/harden the cement. The zinc pyrithione provides a long-term anti-microbial effect and assists in reducing or eliminating any subsequent infections.

Although the present invention has been described with reference to preferred embodiments, numerous modifications and variations can be made and still the result will come within the scope of the invention. The described embodiments are to be considered in all respects only as illustrative and not restrictive. No limitation with respect to the specific embodiments disclosed herein is intended or should be inferred. For instance, while described as a dental cement, the cements of this invention also have applicability in orthopedic settings and can be used to repair or replace bone. Therefore, the scope of the invention is indicated by the appended claims rather than by the foregoing description. All changes which come within the meaning and range of equivalency of the claims are to be embraced within their scope.

What is claimed is:

1. A tooth cement comprising:
at least one barium silicate compound; and,
an anhydrous carrier;
wherein the at least one barium silicate and anhydrous carrier are blended into a non-reactive paste that when mixed with water to form a will form a coalescent paste and harden into a solid mass.

2. The tooth cement of claim 1, the at least one barium silicate compound being chosen from the list of barium silicates consisting of: barium silicate, dibarium silicate, and tribarium silicate.

3. The tooth cement of claim 1, the non-reactive paste being flowable such that it may be stored and administered from a syringe.

4. The tooth cement of claim 1, further comprising an anti-microbial additive.

5. The tooth cement of claim 1, the anti-microbial additive being zinc pyrithione.

6. The tooth cement of claim 5, the zinc pyrithione comprising between .05% to .1% of the cement by weight.

7. The tooth cement of claim 5, the zinc pyrithione comprising between .1% to 1.0% of the cement by weight.

8. The tooth cement of claim 5, the zinc pyrithione comprising between 1.0% to 10.0% of the cement by weight.
